(19) United States
(12) Patent Application Publication
(10) Pub. No.: US 2015/0202097 A1
(43) Pub. Date: Jul. 23, 2015

(54) METHOD AND APPARATUS FOR STACKED CONFIGURATION OF MULTIPLE FEMININE PANTY LINERS

(71) Applicant: Christina M Budde, Delmar, IA (US)

(72) Inventor: Christina M Budde, Delmar, IA (US)

(21) Appl. No.: 14/587,137

(22) Filed: Dec. 31, 2014

Related U.S. Application Data
(60) Provisional application No. 61/929,321, filed on Jan. 20, 2014.

(51) Int. Cl. A61F 13/56; A61F 13/84

(52) U.S. Cl. CPC A61F 13/5611; A61F 13/84; A61F 2013/5683

(57) ABSTRACT

A system is disclosed which provides for a stacked configuration of a plurality of panty liners where a wet wipe is coupled to non-body facing side of at least one of the panty liners and where the center of the non-body facing side is free of any adhesive which is configured to hold said panty liners together.

### FIELD OF THE INVENTION

The present invention generally relates to panty liners, and more particularly relates to stacked panty liners, and, even more particularly, relates to convenient systems and methods for using stacked panty liners.

### BACKGROUND OF THE INVENTION

In the past, it has been proposed to provide a simple plurality of substantially identical panty liners in a stacked configuration so that one can be simply removed and thereby exposing another unused panty liner positioned for immediate use.

While these simple stacked panty liner systems may have many advantages in particular applications, they also have some drawbacks. The secure placement, use inside a garment, and the removal of the top layer panty liner in a stacked configuration of panty liners may be problematic in some situations. The terms top, front, upper and inside are used herein to refer to orientation which is proximal to the body. While bottom, back, lower and outside are used herein to refer to an orientation which is distal of the wearer's body. Additionally, the substantially identical panty liners provide little protection to the edges of bottom or lower layer panty liners.

Consequently, there exists a need for improved methods and apparatuses for deploying multiple panty liners in a stacked configuration.

### SUMMARY OF THE INVENTION

It is an object of the present invention to provide a convenient, clean and efficient system and method for utilizing stacked panty liners.

It is a feature of the present invention to utilize and provide a top level stackable panty liner which is slightly oversized with respect to a next lower level panty liner.

It is an advantage of the present invention to provide a lower level panty liner which is protectively stored while being worn.

It is another feature of the present invention to provide a disposable wipe stored on a panty liner, so as to be accessible immediately upon removal of a panty liner.

It is another advantage of the present invention to reduce the commonly felt preference to change panty liners only in a location having a sink.

It is another feature of the present invention to include non-centrally disposed adhesive dots on a back side of an upper level panty liner.

It is an advantage of the present invention to include adhesion points around a centrally disposed wipe.

The present invention is an apparatus and method for efficiently and hygienically providing a stacked and stored configuration of panty liners, which is designed to satisfy the needs mentioned above, provide the previously stated objects, include the above-listed features, and achieve the advantages explained above. The present invention is carried out in a “hassle free” manner, in a sense that a common level of difficultly in locating an additional panty liner and in cleaning up after a change of a panty liner may be avoided or reduced.

Accordingly, one embodiment of the present invention is directed to a method of deploying a plurality of stacked panty liners. In one embodiment thereof, this method includes the removing of a stored wipe from a backside of a panty liner to be discarded after use.

Additionally, the present invention is a stacked panty liner configuration with increasing sizes of each panty liner as they are stacked on another panty liner affixed inside a wearer's underwear.

The present invention is: A method of changing a panty liner of a person wearing a garment, comprising the steps of:
lowering said garment;
removing a top level panty liner from an adhered configuration with a first lower level panty liner, which first lower level panty liner is at least indirectly coupled to said garment;
removing a wipe from an attached configuration from said top level panty liner;
using said wipe; and
donning said garment so that said first lower level panty liner is configured to be proximal to said person wearing said garment.

### BRIEF DESCRIPTION OF THE DRAWINGS

The invention may be more fully understood by reading the foregoing description of the preferred embodiments of the invention, in conjunction with the appended drawing wherein:

FIG. 1 is an exploded perspective diagram illustrating a two panty liner stacked configuration.

FIG. 2 is another exploded view of the panty liners of FIG. 1, where one panty liner is shown in a slightly rotated orientation while the other panty liner is shown in side view.

FIG. 3 is plain view of a backside of an upper one of the panty liners of the FIG. 1.

FIG. 4 is stacked plain view of the two panty liners of FIGS. 1 and 2.

FIG. 5 is a partially assembled view of the components of the present invention deployed in a wearer's underwear taken from a vantage point inside of the wearer's underwear.

FIG. 6 is another assembled view of the components of FIG. 5, except that the vantage point is from outside the wearer's underwear.

### DETAILED DESCRIPTION

Although described with particular reference to a two panty stacked configuration, the system and method for deploying multiple stacked panty liners can be used in any system that has at least two panty liners in a stacked configuration.

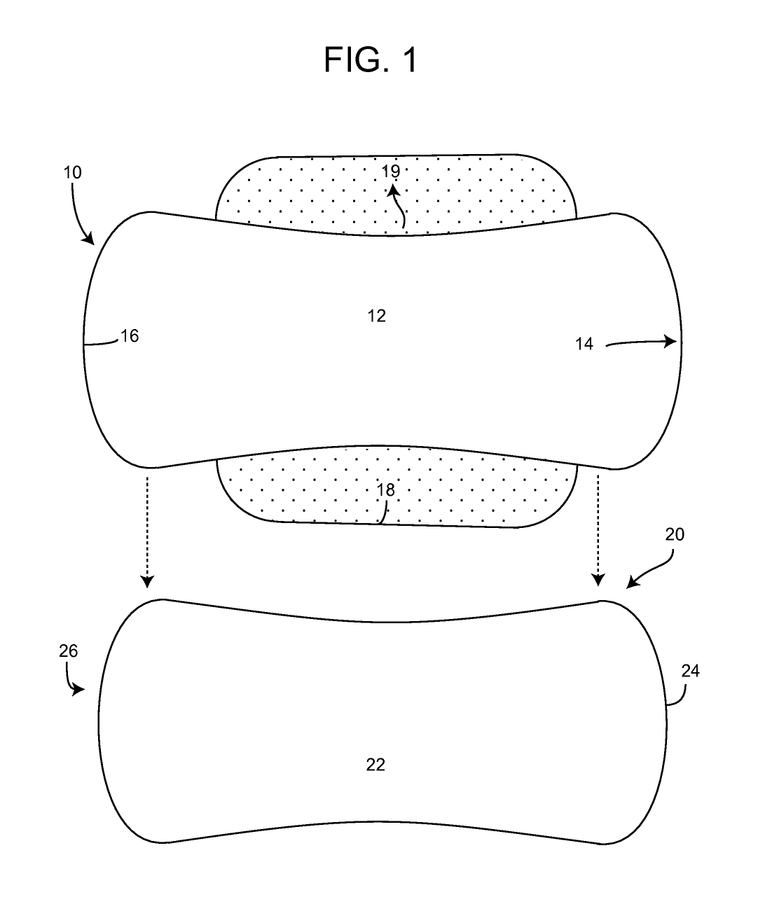

Now referring to FIGS. 1-6, where like numerals refer to like matter throughout, there is shown an example of the present invention. Now referring specifically to FIG. 1, there is shown a top level panty liner 10 with an absorbent body contacting side 12 with a first end 14 and second end 16 and left wing 18 and right wing 19. Also shown is a wingless panty liner 20 having an absorbent body contacting side 22 with a third end 24 and a fourth end 26.

The wingless panty liner 20 will attach like a typical liner to the inside of the underwear. This is the bottom of the stackable liners. In one embodiment, the wingless panty liner 20 may be a prior art commercially available panty liner.

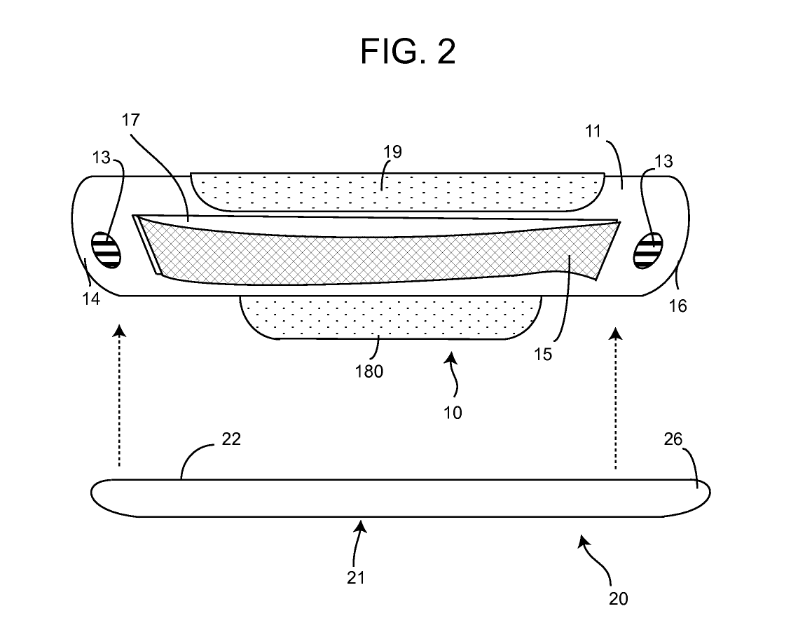

Now referring to FIG. 2, there is shown an alternate perspective view of the panty liners 10 and 20 where an outside (non-body contacting side) 11 is shown as well as some adhesive pads 13. Also shown disposed on backside 11 is a wipe pouch 17 for containing therein a flushable feminine wipe 15. Wipe pouch 17 is intended to be shown as a rectangular pouch, similar to the size and shape of a wrapper for a standard bandage, which shows a flushable feminine wipe 15 therein as if wipe pouch 17 is transparent. Wipe pouch 17 may be foil lined and non-transparent or other suitable design. The absorbent body contacting side 22 of wingless panty liner 20 is shown next to the backside 11 of top level panty liner 10. In this embodiment, wipe pouch 17 may be held in place by adhesive contacting backside 11. Adhesive pads 13 are for adhering the backside 11 to absorbent body contacting side 22 so as to hold them together when top level panty liner 10 is in use and then allow top level panty liner 10 to be pulled away from wingless panty liner 20. In such an arrangement, wingless panty liner 20 with outside 21 is configured with adhesive for attaching to a person's underwear while being worn. Left wing backside 180 of left wing 18 may be covered with adhesive material for contacting the wearer's underwear. Right wing 19 is shown in a slightly folded orientation.

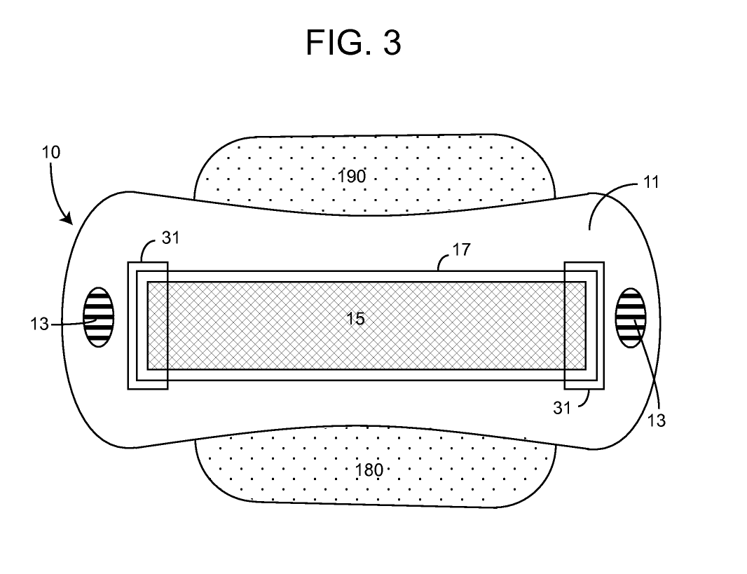

Now referring to FIG. 3, there is shown a close up view of the backside 11 of top level panty liner 10, with an exception that wipe pouch 17 may have no adhesive coupling it to backside 11, because there is shown additional pouch retaining members 31 at each end of wipe pouch 17. These pouch retaining members 31 allow the center of the backside 11 to be free of any adhesive. Pouch retaining members 31 are shown as being transparent for aiding in understanding of the figures but non-transparent members may be preferred in some embodiments. Top level panty liner 10 may be similar to a commercially available consumer feminine panty liner which has been altered by adding the adhesive pads 13 and a wipe pouch 17 with a flushable feminine wipe 15. Backside 11 is shown having right wing backside 190, which may be partially or completely covered with adhesive, similar to left wing backside 180, for adhering to the wearer's underwear.

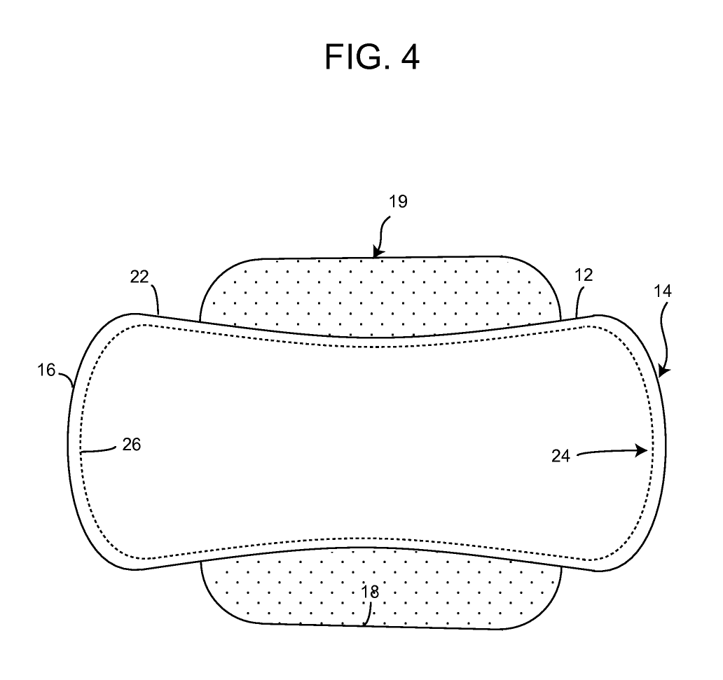

Now referring to FIG. 4, there is shown a view of the panty liners 10 and 20 where the top level panty liner 10 is attached at the backside 11 (not shown) to the absorbent body contacting side 22 (shown outlined by phantom lines) of liner 20. The first end 14 of top level panty liner 10 is shown slightly covering the third end 24 of wingless panty liner 20, shown with phantom lines. In this configuration, it is easily seen that wingless panty liner 20 is protected behind the top level panty liner 10 so as to remain clean until top level panty liner 10 is removed and discarded.

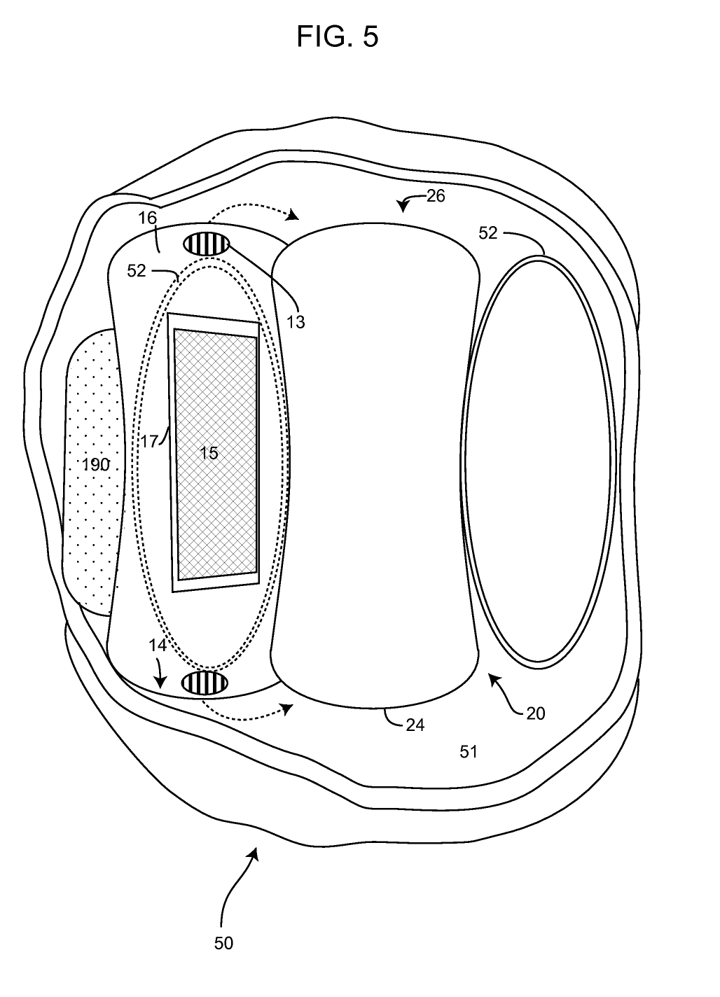

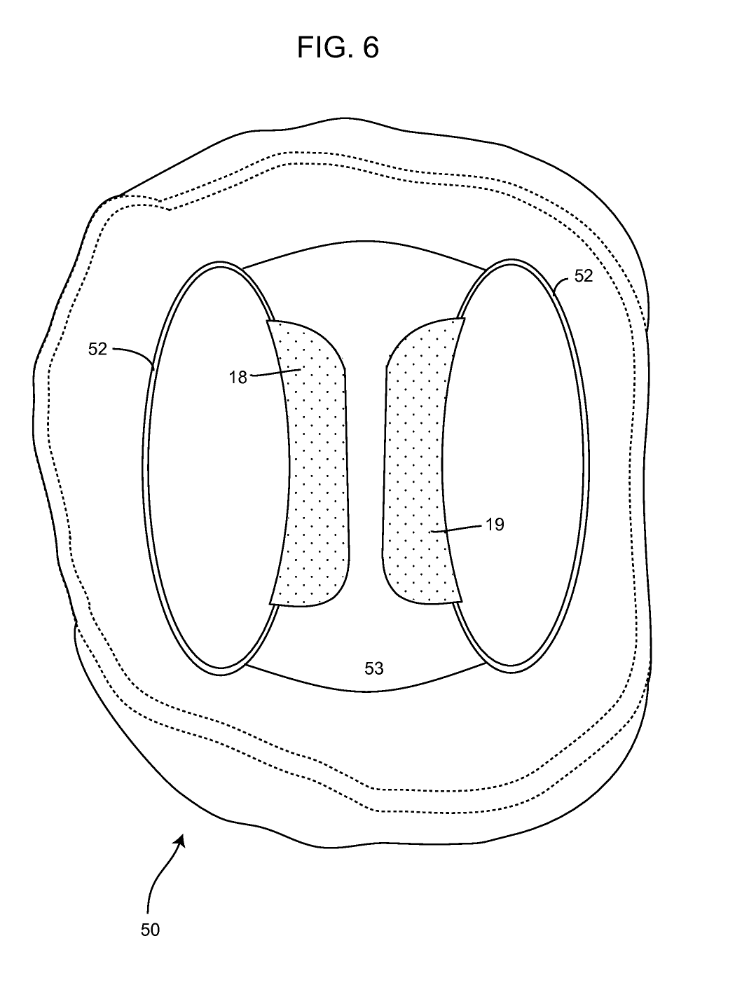

Now referring to FIG. 5, there is shown a garment, such as underpants 50, having an inside body contact surface 51 and a plurality of underpants leg openings 52, one of which is shown with phantom lines as it is obscured by top level panty liner 10. Wingless panty liner 20 is shown attached to the inside body contact surface 51. Top level panty liner 10 is shown positioned after it has had left wing backside 180 (not shown) attached to the underpants outside surface 53 (FIG. 6). The dotted arrows show the direction of rotation of top level panty liner 10 when it is finally secured. When top level panty liner 10 is pivoted, right wing backside 190 will pass through the underpants leg openings 52 (with solid lines) and can be attached to underpants outside surface 53 as shown in FIG. 6. Adhesive pads 13 will adhere top level panty liner 10 to absorbent body contacting side 22 of wingless panty liner 20 when top level panty liner 10 is pivoted over wingless panty liner 20.

Now referring to FIG. 6, there is shown a view of underpants 50 including underpants outside surface 53 with left wing 18 and right wing 19 after they have passed through the underpants leg openings 52 and have been adhered to underpants outside surface 53. Three or more panty liners may be used by adding an additional intermediate liner which is substantially the same as top level panty liner 10, however such an additional liner would be sized slightly smaller than top level panty liner 10 so that it could be covered and protected by top level panty liner 10 and slightly larger than wingless panty liner 20. Alternatively, the stack of three or more could be thought of as including an additional over the top level panty liner which is sized and configured to cover the top level panty liner 10. As many such layers of liners can be used as is comfortable to the wearer.

In operation, the system and method of the present invention can be operated as follows:

Wingless panty liner 20 is placed within a person's underpants 50 as shown in FIG. 5.

Top level panty liner 10 is disposed inside the underpants 50 and the left wing backside 180 with adhesive thereon being exposed is inserted through the underpants leg openings 52 and attached to the underpants outside surface 53. The top level panty liner 10 is then pivoted over the wingless panty liner 20 and the right wing 19 is passed through the other of the underpants leg openings 52 and the right wing backside 190 with adhesive exposed is attached to the underpants 50 forming the configuration as shown in FIG. 6.

If three or more panty liners are desired to be worn at one time, then the process of the immediately preceding paragraph is repeated either identically or by inserting into the underpants 50 a panty liner otherwise identical to the then inner most liner, except that it is sized so as to cover and protect such an inner most liner.

Once the desired number of panty liners are stacked and the underpants 50 is put on by the wearer, the absorbent body contacting side 12 of top level panty liner 10 (or of the inner most liner if three or more panty liners are used at once) is allowed to contact the person wearing the underwear. Then after some time of use, the top level panty liner 10 (or inner most liner if three or more are in place) is removed from the still attached wingless panty liner 20 (or top level panty liner 10 if three liners are used) and flushable feminine wipe 15 is removed from the wipe pouch 17 on the backside 11 of removed liner. The wipe is used and discarded as is the removed liner. The remaining liner(s) is (are) correctly deployed for use without any further steps.

The above system and method describes the process as starting with a wingless panty liner 20 as the first liner to be adhered to the underpants 50. It should be understood that the wingless panty liner 20 could be omitted or the first liner could be any appropriately sized and type of liner including a liner similar to the top level panty liner 10. Depending on the first liner used, the next liner could be an enlarged version of liner described herein as being the top level panty liner 10, except that it is made so as to cover the adjacent lower level liner.

It is thought that the method and apparatus of the present invention will be understood from the foregoing description and that it will be apparent that various changes may be made in the form, construct steps and arrangement of the parts and steps thereof without departing from the spirit and scope of the invention or sacrificing all of their material advantages. The form herein described is merely a preferred exemplary embodiment thereof.

I claim:

1. A method of changing a panty liner of a person wearing a garment, comprising the steps of:
lowering said garment;
removing a top level panty liner from an adhered configuration with a first lower level panty liner, which first lower level panty liner is at least indirectly coupled to said garment;
removing a wipe from an attached configuration from said top level panty liner;
using said wipe; and
donning said garment so that said first lower level panty liner is configured to be proximal to said person wearing said garment.

2. The method of claim 1 wherein said step of removing a top level panty liner is free of any step of breaking any adhesive bond centrally located between said top level panty liner and said first lower level panty liner

3. The method of claim 1 wherein said step of removing a wipe further comprises the step of removing said wipe from a pouch configured to retain said wipe.

4. The method of claim 2 wherein said step of removing a wipe further comprises the step of removing a wipe containing pouch from an adhered configuration with said top level panty liner.

5. The method of claim 4 wherein said step of removing a wipe further comprises extracting said wipe from said wipe containing pouch.

6. The method of claim 5 further comprising the steps of:
lowering said garment;
removing said first lower level panty liner from an adhered configuration with a second lower level panty liner which second lower level panty liner is at least indirectly coupled to said garment;
removing a wipe from an attached configuration from said first lower level panty liner;
using said wipe; and
donning said garment so that said second lower level panty liner is configured to be proximal to said person wearing said garment.

7. A system for storing panty liners for ready deployment within a garment comprising:
a wingless panty liner coupled to said garment;
a top level panty liner disposed over said wingless panty liner so that said top level panty liner covers said wingless panty liner;
said top level panty liner further comprising:
an absorbent body contacting side;
an opposing backside; and
a wipe containing pouch centrally disposed on said backside of said top level panty liner so as to be located between said wingless panty liners and said absorbent body contacting side of said top level panty liner.

8. The system of claim 7 wherein said backside further comprises:
top edge, a bottom edge, a right edge and a left edge and a center; and
wherein said center is free of any adhesive disposed thereon.

9. The system of claim 8 further comprising a plurality of adhesive pads disposed on opposing ends of said wipe containing pouch.

10. A system for storing panty liners for ready deployment within a garment comprising:
a first panty liner coupled to said garment;
an upper level panty liner disposed over said first panty liner so that said upper level panty liner covers said first panty liner;
said upper level panty liner further comprising:
an absorbent body contacting side;
a backside, opposing said absorbent body contacting side;
wherein said backside further comprises:
a top edge, a bottom edge, a right edge and a left edge and a center; and
wherein said center is free of any adhesive disposed thereon;
a wipe containing pouch centrally disposed on said backside of said upper level panty liner so as to be located between said first panty liners and said absorbent body contacting side of said top level panty liner.

11. The system of claim 10 wherein said wipe containing pouch is detachably coupled to said back side via a plurality of pouch retaining members.

12. The system of claim 11 wherein a wipe can be removed from said wipe containing pouch by tearing said pouch.

13. The system of claim 10 wherein a wipe can be removed from said wipe containing pouch by tearing said pouch.
